(19) United States
(12) Patent Application Publication
(10) Pub. No.: US 2010/0168869 A1
(43) Pub. Date: Jul. 1, 2010

(54) Tissue integration implant

(75) Inventors: Marc Long, Denville, NJ (US); Twana Davisson, Montclair, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 12/319,138

(22) Filed: Dec. 31, 2008

(51) Int. Cl. A61F 2/02

(52) U.S. Cl. 623/23.72

(57) ABSTRACT

The present invention relates to a tissue defect repair implant including a porous material, wherein the material is expandable or compressible. The implant may be a scaffold, and may also be formed of a material such as, for example, collagen or demineralized bone matrix. Another aspect of the invention may be a method for the repair of a tissue defect in a patient in need thereof, including implanting an implant between two adjacent tissues, wherein the implant may include a porous material which is expandable or compressible. The two tissues may be soft tissue and hard tissue, and alternatively, the two tissues may be soft tissue, and may further be the same or different soft tissues.

### FIELD OF THE INVENTION

The present invention relates to the field of medical technology and is generally directed to the treatment of various tissue defects through the use of grafts and fillers. The treatment may be directed to tissue such as ligaments, tendons, cartilage, connective tissues, or the like, as well as integration of connective tissues to bone, soft tissue to soft tissue, soft tissue to hard tissue, bone to bone, or the like.

### BACKGROUND OF THE INVENTION

Soft tissue, such as ligaments, tendons, muscles, cartilage, meniscus, fibrous tissues and other like soft connective tissues, connect, support and surround other parts of the body such as hard tissue, for example, bone. Soft connective tissues are generally made up of the protein collagen, and in the case of ligaments and tendons, the collagen fibers are densely packed to provide a high tensile strength.

Soft tissues can be damaged due to traumatic injury, and wear and tear due to age, disease or lifestyle conditions. Damage to such soft tissues can cause joint instability and severe pain. These soft tissues have limited regenerative capabilities on their own, and thus require normally invasive techniques in order to repair the trauma.

Further, damage to a soft tissue, such as a ligament, at the connection site to the bone can cause additional trauma to the bone itself. These types of injuries can be especially difficult to treat as the connection site has been weakened by the injury. Typical treatments, such as screws and anchors, may produce poor results due to the lack of strength of the connection site following the injury.

One critical issue is the limited contact area, or “footprint,” between the tissues to be repaired with current repair techniques. For example, sutures only cover a small area of the tissue being repaired and the consequent area in contact between the tissues to be repaired is limited. Subsequently, the integration between tissues and the resulting repair, or lack thereof, is poor. There is a need in the current state of the art to augment mechanical repairs, provided by the current state of the art, with a biological repair piece, where the contact area between tissues may be maximized to produce enhanced healing.

Similarly, cartilage is an avascular soft connective tissue made up of collagen and/or elastin fibers, and chondrocytes, all of which are embedded in a matrix. There are three main types of cartilage: elastic, fibrocartilage, and hyaline. Elastic cartilage is found in the outer ear and the epiglottis. Fibrocartilage is found between the bones of the spinal column, hips and pelvis. Hyaline cartilage can be found on the ends of bones which form joints, on the ends of the ribs, on the end of the nose, on the stiff rings around the windpipe, and supporting the larynx. Articular cartilage is a specialized type of hyaline cartilage which covers the surface of joints and provides a durable low friction surface that distributes mechanical forces and protects the joint's underlying bone.

Loss of or damage to cartilage can lead to painful conditions such as osteoarthritis. Damage to cartilage, like other soft tissues, can be caused, by traumatic injury, disease and/or age. Since cartilage lacks nerves and blood vessels, it has very limited regenerative capabilities compared to other tissues. Consequently, the healing of damaged joint cartilage results in a fibrocartilaginous repair tissue that lacks the structure and biomechanical properties of normal cartilage. Over time, the repair tissue degrades and leaves damaged joint cartilage, which causes osteoarthritis and reduced movement in the joint.

Similar to other soft tissues, as discussed above, cartilage-bone connection sites can also experience trauma which is difficult to treat and heal properly. As above, current methods of treating cartilage-bone connection sites produce poor results which do not produce a properly healed connection site.

There is a need for devices and methods for repairing soft tissue defects, as well as a need for methods for repairing connection sites of soft tissue to bone.

### SUMMARY OF THE INVENTION

One aspect of the invention may be a tissue defect repair implant including a porous material, wherein the material is expandable or compressible. Further, the implant may be a scaffold, may allow for tissue ingrowth, and may also be formed of a material such as, for example, collagen or demineralized bone matrix.

Another aspect of the invention may be a method for the repair of a tissue defect in a patient in need thereof, including implanting an implant between two adjacent tissues, wherein the implant may include a porous material which is expandable or compressible. A tissue defect, for example, may be within either one of the two tissues, or may be the site where the two tissue detached from one another. The implant may further be implanted between the two tissues to enhance integration between the two adjacent tissues, through the implant, such that the two tissues may attach to one another.

The two tissues may comprise bone and a tissue selected from, for example, tendons, ligaments, fibrous tissues, meniscus tissue, labrum, muscle, cartilage, skin, dermal layers, joint capsule and synovium, bone, and the like. Alternatively, the two tissues may be tissue other than bone, and may further be the same tissue, such as for example, cartilage.

As to tendon and ligament repair, for example, the implant may also be used for defects such as reattachment of the soft tissue to bone at the original attachment site of the soft tissue to the bone, or for reattachment of two pieces of tendon or ligament which had been severed.

### BRIEF DESCRIPTION OF THE DRAWINGS

FIG. 1. shows various arrangements of the implant, including one arrangement which has an additional material, illustrated here as a graft.

FIG. 2. shows various embodiments of the implant of the present invention in which the implant and graft are arranged in different configurations for integration of soft tissue to hard tissue.

FIG. 3. shows yet another embodiment of the implant used for integration of a soft tissue to a hard tissue.

FIG. 4 shows a further embodiment of the implant used for integration of both a partial-thickness tear and a full-thickness tear of the rotator cuff.

FIGS. 5a-c shows yet other embodiments of the implant used for integration of soft tissue (e.g., ACL ligament) within a bone tunnel.

### DETAILED DESCRIPTION OF THE INVENTION

The present invention is directed to the repair of various soft tissues and includes a tissue implant and a method of repairing a tissue defect using the implant.

As used herein, the term “soft tissue” relates to tissues that connect or support structures within the body, such as, for example, tendons, ligaments, fibrous tissues, meniscus tissue, muscle, labrum, cartilage, skin, dermal layers, joint capsule, synovium, and the like.

As used herein, the term “hard tissue” is used to refer to bone, calcified tissue, or the like.

In many of the illustrative examples described below, cartilage and other specific soft tissues will be used to exemplify various embodiments of the invention. These examples are not meant to limit the scope of the invention in any way, as the methods and devices disclosed herein may be used with other soft tissues.

Although a number of different therapeutic methods are currently being used to treat soft tissue defects, they have only been marginally successful. Some of the current treatments, for example, specific to the repair of cartilage and like tissues include lavage, arthroscopic debridement, and repair stimulation. However, these therapeutic methods either provide only temporary pain relief or have shown limited clinical efficacy.

Other treatment methods involve grafting the defect site with artificial materials, autografts, allografts, or xenografts. Examples of different grafts and grafting methods can be found in U.S. patent and application U.S. Pat. Nos. 5,944,755; 5,782,915; 6,858,042; 2003/0229400; and 2004/0230303, the disclosures of which are incorporated by reference herein. Grafts for tissue repair include porous materials, such as PLA, collagen “sponges”, hyaluronic acid, metals (CoCr, Titanium), PVA, autograft, allograft osteochondral plugs, and xenografts such as bovine collagen. None of these materials are both porous and expandable or compressible to a significant amount of their original size.

One particular grafting method for the repair of various tissue defects, called mosaicplasty, has shown some clinical efficacy. Mosaicplasty involves removing small autologous osteochondral plugs from low weight bearing sites in a patient's joint. The osteochondral plugs are then grafted into a mosaic of holes drilled into the patient's articular cartilage defect site. Some patients who have undergone mosaicplasty have reported decreased pain and improved joint function. Marcacci, M. et al., Arthroscopy 21(4): 462-470 (2005).

Although all of the above methods have had some clinical success, each one of these therapeutic methods suffer from one or more of the following disadvantages: the risk of patient immune response or disease transmission; limited availability of osteochondral autograft sites; lack of implant adhesion to the defect site; implant deterioration; lack of long-term efficacy; donor site morbidity; patient discomfort; and the failure to restore normal joint function.

The Osteosponge™ (Bacterin International, Inc.; Belgrade, Mont.) has been developed for bone defects. It is a porous, compressible and expandable demineralized bone matrix (DBM), which has been shown to be useful as a scaffold for bone repair. The present inventors have shown that the Osteosponge™ can also be used for soft tissue repair. The compressible and expandable DBM sponge is porous and can be compressed to 30% of its size prior to implantation. See the following U.S. Publications and Issued Patents for similar products: 2006/0085075; 2005/0090899; 2004/0115240; 2004/0197375; 20040062753; 20040166169; 20040078090; U.S. Pat. Nos. 7,056,337; 6,121,042; 6,319,712; 6,171,610; 5,882,929; and 6,124,273, the disclosures of which are hereby incorporated herein by reference, as if fully set forth herein.

As shown in U.S. Application Publication No. 2008/0039954, the disclosure of which is hereby incorporated by reference herein as if fully set forth herein, a graft comprising a material with sponge-like properties similar to Osteosponge™ is used in the repair of cartilage defects (chondral or osteochondral). The graft allows, in one example, cartilage growth, resulting in restoration of function. Also disclosed was a method for cartilage repair comprising implanting a graft into a cartilage defect in a patient, wherein the graft comprises a porous material which is also expandable and/or compressible. The material is porous, to allow in-growth of cells. The material is also compressible and/or expandable for better press-fit and chondro-integration.

A subsequent application, filed concurrently herewith, U.S. patent application Ser. No. ______ , entitled “Multiple Piece Tissue Void Filler,” the disclosure of which is hereby incorporated by reference herein as if fully set forth herein, discloses an implant which includes a scaffold similar to Osteosponge™ which is used to repair cartilage defects. The scaffold can, in one embodiment, be wrapped around a graft, which provides both a tighter press-fit of the graft into the defect, as well as providing a scaffold through which tissue in-growth can occur to integrate the graft into the defect. Alternatively, a cartilage defect can be packed with the scaffold to promote cartilage growth within the defect. The inventors have also discovered that this material may also be used in the integration of soft tissues to soft tissues, either the same or different soft tissues, soft tissue to hard tissue, and the like.

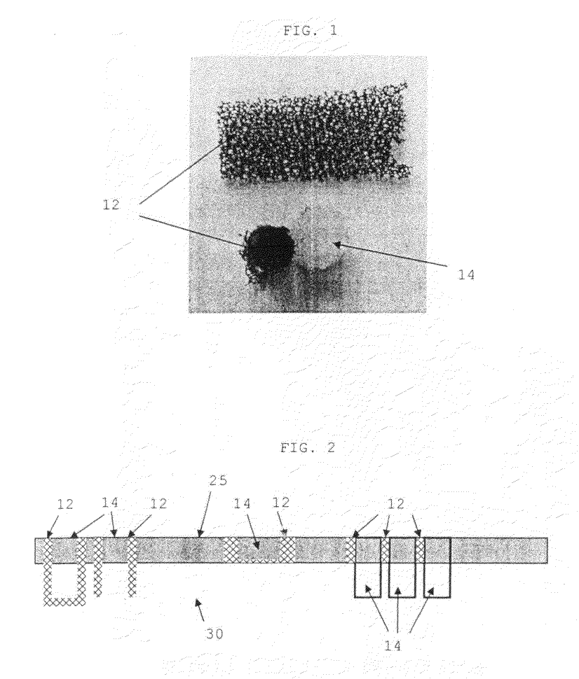

As illustrated in FIG. 1, in one embodiment of the present invention, an implant 12 may include a material which may be a scaffold which may be expandable and/or compressible. The implant may be in various shapes, including, but not limited to, cylindrical, flat sheet, rectangular, square-shaped, hexagonal, spherical, conical, X-shaped, T-shaped, tear-drop shaped, trapezoidal, or the like. Specifically, the scaffold of the implant may be in any shape required to fill a tissue defect or void completely. In FIG. 1, the implant 12 is illustrated to be both a flat sheet and a cylindrical shape. The cylindrical shape of the implant 12 may be, for example, a flat sheet rolled on itself, or it may be formed directly into the shape of a cylinder. Likewise, the flat sheet shape may be folded on itself to form an oblong or oval shape.

In one embodiment, the implant 12 includes a scaffold which is made of DBM, and may further specifically be similar to OsteoSponge™. The DBM is porous, compressible, and/or expandable, and is thus suitable for a tight press-fit application. The DBM may also be compressible and/or expandable in all dimensions, thus creating a more malleable material which can be used in a variety of locations and applications.

In larger tissue defects, such as those over 1 cm², the implant 12 may be combined with an additional material 14. The implant 12 and additional material 14 may be made from the same material or from different materials, and may be the same shape or different shapes. In one embodiment, the additional material 14 may be any type of graft, some examples of which were mentioned above, which may be found currently in the art.

Both the implant 12 and the additional material 14 may be composed of synthetic, natural, or recombinant material, or any combination thereof. The natural material may be of human, animal, and/or plant origin such as, for example, silk, collagen or hyaluronan-based material or the like. The synthetic material may be, for example, silk or a resorbable polymer, or a co-polymer, from the family of, for example, polycaprolactone, polyurethane, polyester, polyethylene, or the like, or a hyaluronan-based material. One naturally derived material which may be useful in the invention is DBM. The recombinant material may be collagen or silk.

In one embodiment, the scaffold may be a mesh structure. The mesh may include any of the materials above.

In another embodiment, the scaffold may be a 3D woven scaffold composed of woven fibers of various materials to create a 3D fibrous architecture beneficial to cell and tissue ingrowth. One such architecture may be for example that described in “A biomimetic three-dimensional woven composite scaffold for functional tissue engineering of cartilage,” Moutos, Freed, and Guilak in Nature Materials, Vol. 6, February 2007, pp. 162-167.

In still other embodiments, the scaffold may be of biomimetic nature, such that it ressembles or mimics a natural tissue architecture found in a natural organism. One example is the DBM architecture of the OsteoSponge™. Another example is a xenograft material derived from animal tissue. One such scaffold is TissueMend™ (TEI, Boston, Mass.; Howmedica Osteonics Corp., Mahwah, N.J.), which is made of fetal bovine dermal. Another such scaffold maybe equine pericardium (e.g., from Pegasus Biologics, Irvine, Calif.), porcine dermis (e.g., from SIS Depuy Mitek, Raynham, Mass.), or the like.

In certain embodiments, the scaffold structure of implant 12 is made of DBM, and the DBM may be processed to allow for variations in degree of demineralization throughout the implant 12. This may affect the compressible/expandable nature of the implant 12, so that its compressible nature may vary with location in both structures. This may be particularly advantageous in reconstructive procedures where structural rigidity of an implant is imperative. Of course, this degree of demineralization may also be present in the additional material 14, or in multiple implants 12 which may be implanted into the same defect or other implantation site. The degree of demineralization of each individual implant 12 and/or any additional materials 14 may be the same or may vary from one another.

For example, a devitalized cartilage matrix may be produced using a process similar to that used to create Osteosponge™. The starting material could be either cartilage only or could be an osteochondral core. Any source of cartilage cells could be used. Either could be processed to achieve a material for implant 12, or additional material 14, that is expandable and/or compressible and appropriate for cartilage repair.

Besides the porous material for soft tissue integration and/or growth, the implant may, in one embodiment, include other portions, for example, a bone portion. The implant may consist, for example, of a cartilage portion extending into the bone portion of the defect. The implant may also consist of a bone portion extending into the cartilage portion of the defect. Alternatively, the implant 12 may consist of two separate pieces used in the same defect; a cartilage-appropriate portion and a separate bone-appropiate portion. The two portions may also be separated by a membrane to prevent fluid migration or may be used as delivery of biological factors.

Similarly, within the implant 12, the degree of demineralization may change, creating a demineralization gradient through at least a portion of the implant 12, through multiple implants 12, and/or through additional material 14. For example, as to the implant 12, the degree of demineralization may form a demineralization gradient throughout the volume of material. In one embodiment, the gradient may occur from the bottom of the implant 12 to the top. In another embodiment, the gradient may occur from the interior of the implant 12 to the external surface. In a further embodiment, the gradient may differ from one side of the sheet to the other side of the sheet. The gradient, in one embodiment, may change in an axial or radial direction. A gradient such as these described would allow a single piece of implant 12 to have both higher strength properties in one portion of implant 12 and higher porosity, tissue ingrowth, compressibility and expandability in another portion of implant 12.

Additionally, more than one implant 12 may be placed within a single defect site. For example, if multiple implants 12 are placed within a single defect site, all of which are made of DBM, the implants may conform to each other to create intimate contact between each implant and to the surrounding tissue. This intimate contact may be generally continuous throughout the volume of the defect site and may further substantially fill the defect site, thus providing contact healing throughout the defect site along with a scaffold throughout which tissue may be regenerated. The multiple implants may be similar in shape to each other, or may be differently shaped from one another.

The implant 12 may, in some embodiments, be implanted into a defect site once the defect has been identified. The defect may first be cleaned, debrided and prepared. Tools may be used to form the defect site into a cylindrical shape, or alternatively into another shape such as an oval, square, rectangle, or any other shape. In the case where a multiple piece implant, or multiple implants, is used, one piece may be added at a time into the defect. Once the first piece is implanted, it may be compressed, for example, radially, to make room for the implantation of a second piece. Once the second piece is within the defect, the first piece may be released, thus allowing the first and second pieces of implant to come into intimate contact with one another and with the surrounding tissue. This method may be repeated as necessary until the entire defect is substantially filled. Alternatively, if a strip piece is used, it may be rolled up and compressed during insertion into the defect, and once within the defect, it may be released to intimately contact the surrounding tissue such that it substantially fills the defect and conforms to the shape and size of the defect.

Integration with the surrounding soft tissue 25 is not commonly achieved when a typical, known “press-fit” plug is used. A tighter press-fit may be achieved by the expansion of the implant 12 of the invention inside the defect, and will enhance integration and improve the performance of the implant.

The implant 12 of the present invention achieves better apposition with the surrounding tissue and decreases, or eliminates, micromotion. These results would be expected to yield improved healing of the tissue defect and increased longevity. In addition, the implant may provide a scaffold with improved fixation due to its ability to be compressed and expand inside the defect.

The material of implant 12 should be porous enough to allow cell growth. Each pore may be the same size, or the pores may be of varying sizes, so long as some of the pores are large enough to allow cell growth into the material. Additionally, the pores may vary or change in size on compression and/or expansion of the material. In certain embodiments, the material has pores with a diameter of at least about 10 microns, at least about 20 microns, at least about 30 microns, at least about 40 microns or at least about 50 microns. Larger size pores are also within the scope of the invention, for example at least about 75-1000 microns.

The material of implant 12 used in the invention is expandable and/or compressible by a significant amount. By “expandable by a significant amount” it is meant that the material expands by at least about 5 or 10% of its original size by volume. By “compressible by a significant amount” it is meant that the material compress by at least about 5 or 10% of its original size by volume.

In another embodiment, the implant 12 may expand by at least about 5 or 10% to at least about 300% of its original size. For example, the material may expand by at least about 5%, 10%, 20%, 25%, 30%, 50%, 75%, 100%, 150%, 200%, 250%, or 300% of their original size. Likewise, the implant may compress by at least about 10% to at least about 99% of its original size. For example, the material may compress by at least about 5%, 10%, 20%, 25%, 30%, 50%, 75%, or 99% of their original size.

The implant 12 may be bioresorbable, or non-resorbable. While non-resorbable implants may necessitate the need for an additional operative procedure, clinician control over the duration of time the implant remains intact could allow for increased integration of the implant into the defect site. The implant could be constructed to remain implanted for an indefinite period of time without negatively interfering in any biological processes or causing the patient pain.

The implant may be seeded with one or more types of cells prior to, at the time of, or after implantation. “Seeding” the implant with cells refers to the process of inserting, or placing, one or more types of cells into, or onto, at least a portion of the implant. The cells can be placed in or on the porous material of the implant, and can be placed on only one piece of the implant, a portion of one piece of the implant, or on the entire implant or any combination thereof. Likewise, different types of cells can be placed into different areas of the implant depending on the desire of the surgeon.

Suitable cells for seeding the implant include any kind of tissue producing cells, or any kind of cells which may have a therapeutic affect, either in the implant or by migration out of the implant. Suitable cells include, but are not limited to embryonic stem cells, stem cells, bone marrow cells, mesenchymal cells, progenitor cells, synovium cells, synovial fluid cells, chondroblasts, chondrocytes, osteoblasts, or combinations of these cells.

Any cells added to the implant can be retrieved from various sources, including the patient to be treated, other patients of the same species, pools of cells from other patients or animals, individual animals and commercially available cell lines. Cells may be unaltered and seeded onto implants immediately after removal from the source or remain in culture until being added to the implant. The cells may be allogenic, autogenic, or xenogenic to the patient to be treated. Combinations of cells may be used.

The implant may be used as an ex vivo matrix for cell growth and/or may be implanted in situ into a, for example, cartilage defect as an in vivo matrix for cell growth. The invention also comprises an implant produced by culturing with cells.

The implant may be cultured with appropriate cells ex vivo until the appropriate tissue forms and is then implanted, cultured with appropriate cells ex vivo and implanted before full tissue formation, or implanted without any culturing step at all.

One or more biological agents may be added to the implant, a piece of the implant, a portion of a piece of the implant or a portion of the implant. Likewise, different biological agents may be placed in various portions of the implant or may be placed simultaneously in various portions of the implant. By “biological agent” it is meant any agent that has, or produces, biological, physiological and/or pharmaceutical activity upon administration to a living organism. These biological agents may be added to the implant at any time, for example, before, during or after implantation.

The implant can have varying degrees of biological agent content. The presence of biological agents can be controlled such that growth factor content is maximal or negligible. Biological agent content may vary with depth or location.

Suitable biological agents include, but are not limited to, growth factors, cytokines, antibiotics, antimicrobials, biomolecules, drugs, strontium salts, fluoride salts, calcium salts, sodium salts, bone morphogenetic factors, chemotherapeutic agents, angiogenic factors, anti-inflammatory compounds, such as for example IL-1Ra or TNF-alpha, osteoconductive, osteoinductive and osteogenic agents, chondroconductive, chondroinductive and chondrogenic agents, fibroconductive, fibroinductive and fibrogenic agents, conductive, inductive and promotive agents for other soft tissues such as tendon, inductive agents, bisphosphonates, painkillers, proteins, peptides, or combinations thereof. Other biological agents may include cells such as for example allogenic cells, autologous cells, progenitor cells, stem cells, bone marrow stromal cells, mesenchymal cells, fibroblasts, chondrocytes, tenocytes, synovicytes, or the like. Further biological agents may include platelet-rich-plasma (PRP), platelet concentrate, bone marrow concentrate, plasma concentrate, blood, bone marrow, synovial fluid, hyaluronan and hyaluronic acid.

Growth factors that can be added to the implant include platelet derived growth factor (PDGF), transforming growth factor beta (TGFβ), insulin-related growth factor-I (IGF-I), insulin-related growth factor II(IGF-II), beta-2-microglobulin, bone morphogenetic protein (BMP), fibroblast growth factor (FGF), hepatocyte growth factor (HGF), cartilage derived morphogenetic protein (CD-MP), growth differentiation factors (GDFs), or combinations of growth factors.

Chondroinductive agents include prostaglandin E2, thyroid hormone, dihydroxy vitamin D, ascorbic acid, dexamethasone, staurosporine, dibutyrl cAMP, concavalin A, vanadate, FK506, or combinations of different chondroinductive agents. Antibiotics include tetracycline hydrochloride, vancomycin, cephalosporins, and aminoglycocides such as tobramycin, gentamicin, and combinations thereof. Pain killers include lidocaine hydrochloride, bipivacaine hydrochloride, ketorolac tromethamine and other non-steroidal anti-inflammatory drugs.

The biological agent added to the implant may also be a protein or combinations of proteins. For example, proteins of demineralized bone, bone protein (BP), bone morphogenetic protein (BMP), such as BMP-7, osteonectin, osteocalcin, osteogenin, or combinations of these proteins can be added to the implant.

Other suitable biological agents include cis-platinum, ifosfamide, methotrexate, doxorubicin hydrochloride, or combinations thereof.

Other materials such as gels, putties, cements or the like may also be added to the implant. Such materials, for example, may assist in securing the implant in place or to create separations between different pieces of the implant.

The above materials, biologics and cells may also be placed in between the multiple implant pieces which may enhance integration between the multiple pieces and the surrounding tissue.

The implant can be implanted dry or hydrated with liquids before, during, or after implantation. Examples of liquids include, but are not limited to water, saline, and bodily fluids (such as blood, bone marrow or synovial fluid). All or only part of the implant (for example, the porous material or part thereof) may be hydrated. The hydration may be done by any method, including dipping, sprinkling, full or partial submersion, running under a faucet, centrifugation through the scaffold, pressure, vacuum or negative pressure. The implant may be exposed to the liquid for an instant or up to several hours or several weeks, and can be stored in a liquid indefinitely until implantation.

The method of the invention can be used to treat any tissue defect, whether it is in ligament, tendon, meniscus, labrum, elastic cartilage, fibrocartilage, hyaline cartilage, or the like. The method may assist in the repair of tissue defects in order to relieve pain and help prevent the onset, or to delay the progression, of osteoarthritis, for example.

In another embodiment, the implant may also be used when a defect is identified prior to or during a joint repair procedure. For example, the method could be used for cartilage repair in joints, such as a knee, ankle, hip, shoulder, elbow, temporomandibular, sternoclavicular, zygapophyseal, and wrist; or any other place where cartilage is found, such as the ear, nose, ribs, spinal column, pelvis, epiglottis, larynx, and windpipe. The implant may also be used in rhinoplasty procedures, including but not limited to reconstruction via a dorsal septal graft. The implant may be used to repair cartilage during a microtia-atresia surgical correction or in other types of auricular reconstructive procedures, such as those secondary to trauma or cancer.

The implant can be used to repair tissue in any patient in need thereof. By “patient” is meant any organism which has connective tissues, including, but not limited to humans, monkeys, horses, goats, dogs, cats, and rodents.

One implant may be used alone to fill the defect, or multiple implants may be combined to fill one defect (similar to the mosaicplasty technique). In addition, the implant may be used to compliment other tissue repair procedures, including autograft, allograft, or mosaicplasty procedures. The implant of the invention may be implanted at the same time, before, or after other tissue repair procedures.

The expandable/compressible material may be used to fill small gaps left during the other procedures. The implant can be used to fill either the donor or the recipient sites in mosaicplasty-like procedures, and can be used either alone or in combination with other materials, including allografts, autografts, other biomaterials or other grafts. For example, the implant 12 may be a DBM scaffold which is porous, compressible and expandable, while an additional material 14 may be an allograft or autograft. The implant 12 integrates the graft, additional material 14, with the surrounding tissues.

A surgeon may alter the size of the implant material prior to implantation by means of scissors or some other instrument or device used for cutting. This gives the clinician the operative flexibility to customize the fit of the invention without detriment to the patient or the implant itself.

Prior to, after, or in the absence of compression, the implant can be shaped by the clinician to match any anatomical intricacies of the surgical implantation site. The implant can then be implanted, either dry or hydrated, via a procedure such as “press fit.” The implant can be compressed prior to implantation, or can be implanted without compression. The implant material may expand to substantially fill the defect after implantation.

An undersized void can be created in the tissue and possibly the adjacent bone where a defect is identified. For articulating joints, for example, the surgeon may create a defined defect in the articulating joint where fibrillation or a cartilage defect was identified. The defect may be chondral or osteochondral.

The implant 12, which can be oversized compared to the defect, may be compressed and implanted into the defect, either dry or hydrated. The implant may be compressed by any method, including by hand, by squeezing through a conical tube of a desired size, or via surgical instrument.

The implant may fill any void space by expanding to substantially fill the total volume of the defect. The constraint created by the undersized defect creates an increased press-fit with the surrounding tissue, enhanced integration and the elimination of micromotion. The implant may also be implanted without a press-fit or interference fit but will expand after implantation due to hydration with body fluids.

The implant may be merely press fit into the defect area or an anchor can be used to affix the implant to the defect. Anchors include plates, nails, screws, pins, tacks, adhesives, organic glues (such as fibrin glue), clotting materials or any other material known to be suitable for affixing soft tissue and/or bone grafts. More than one type of anchor may be used to affix the implant to the tissue defect site. Anchors such as these may be particularly useful for implantation of the implant into the meniscus to ensure a strong, tight fit to the underlying hard tissue adjacent the meniscus.

Because the implant can be compressible in all dimensions, it can be compressed to fit into small articulating joints, such as the hip. Thus, the ability to be compressed in three dimensions allows an implant to be used in the repair of tissue defects of the hip or other articulating joints or during arthroscopic surgeries.

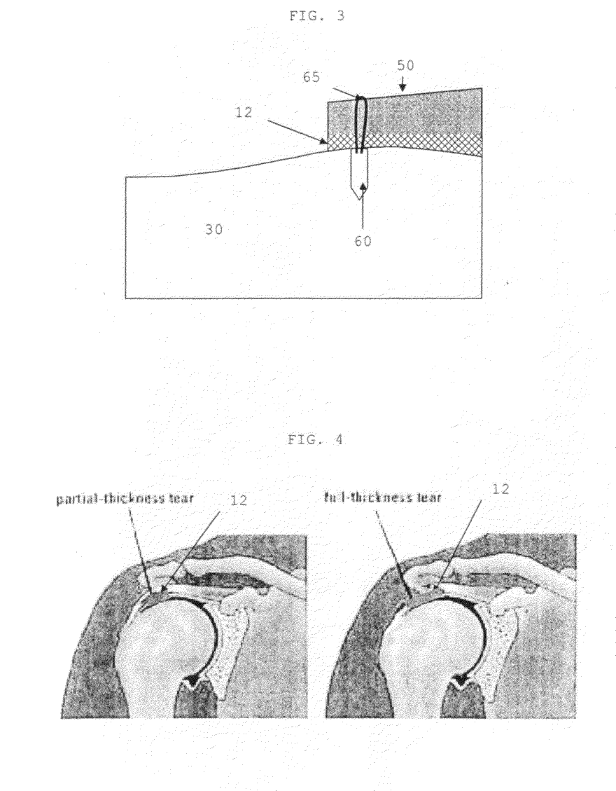

In yet another embodiment, the method of implantation may include integrating two tissues with the implant. Alternatively, an implant 12, which may be made from DBM, may assist in integrating additional material 14, which may be a tissue graft. FIG. 2 illustrates various examples of tissue integration using this method. The implant 12, which may be made from DBM, may be a continuous shape, a wrap (or a sheet), or filler (i.e., chunks). The implant 12, which may be a porous scaffold, allows for tissue ingrowth from the surrounding tissues. The implant 12 is in intimate contact with the additional material 14 as well, and thus the tissue ingrowth may securely fix the implant 12 and the additional material 14 in the tissue defect.

On the left hand side of FIG. 2, implant 12 may surround an additional material 14, which may be osteocondral graft (either allograft or autograft, for example). The implant 12 may be a DBM scaffold, and may assist in integrating the osteocondral graft with the surrounding cartilage 25 and the hard tissue 30.

The middle portion of FIG. 2 illustrates an implant 12, which may be a DBM scaffold, surrounding an additional material 14, which may be, for example, a cartilage graft. The DBM scaffold may assist the cartilage graft in integrating with the surrounding cartilage material. Additionally, the bottom portion of DBM may assist in integrating the cartilage graft with the hard tissue 30 surface.

The right hand portion of FIG. 2 illustrates a series of additional materials 14, which may be mosaicplasty cores, with a series of implants 12, which may be DBM scaffolds, in between each core and the surrounding cartilage 25. As above, the DBM assists in integrating the cores with one another, and with the cartilage 25 surrounding the defects. The cores may include a portion of hard tissue which may provide better integration with the hard tissue 30 at the defect site.

This method of integration may also be used in the integration of soft tissues to each other or to hard tissues, such as bone. For example, integration of bone to ligament, tendon, meniscus, rotator cuff or other soft tissues may occur. Likewise, integration of, for example, tendon to muscle may also occur.

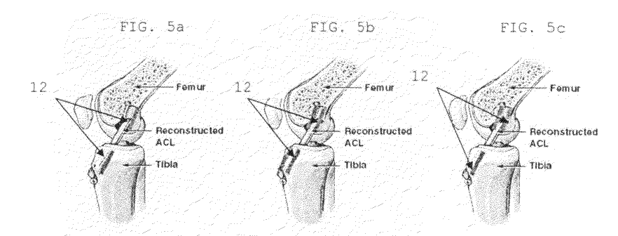

For example, FIGS. 3 and 4 illustrate this method through the integration, within a defect, of the rotator cuff 50 to bone 30. An implant 12, in the form of a strip, which may be DBM scaffold, is placed between the rotator cuff 50 and bone 30 to repair the tissue defect of the rotator cuff separating from the bone. The rotator cuff 50 may also be attached to the bone 30 using traditional techniques such as a suture 65 and suture anchor 60. The implant 12 is securely held in place once the rotator cuff 50 is attached to the bone 30 through the suture 65, thus providing additional assistance in integrating the rotator cuff 50 to the bone 30. Implant 12 may be used to integrate the rotator cuff through reattachment at the original attachment site on the bone, or may integrate the rotator cuff with the bone at a new attachment site.

Similarly, the implant 12 may be placed within the rotator cuff 50, or other soft tissue, to substantially fill a tissue defect within the soft tissue.

Implant 12 may be used in other situations in which there is a soft tissue defect. For example, in the case of a torn anterior cruciate ligament (ACL), illustrated in FIGS. 5a-c, the ligament may be reattached to the bone, at the original attachment site or at a new attachment site, using implant 12. Implant 12 may be wrapped around the ligament, whether it is the existing ligament, allograft, artificial ligament, or the like, and reattached to the bone using an interference screw known in the art. FIGS. 5a-c show various embodiments of the location of implant 12 within, for example, bone tunnels in the tibia and femur, to reattach and secure the ligament to the bone. The implant 12 may also be wrapped around the bone or bone tunnel. The implant 12 promotes integration of the bone and ligament.

Another example may be at a meniscus tear, or an area missing part of the meniscus, in which the implant 12 may be added to integrate the surrounding meniscus to each other, or to the adjacent bone.

A further example may be to use implant 12 on an Achilles tendon tear. In one embodiment, the two pieces of the tendon may be sutured together, then wrapped with implant 12 to promote integration. Alternatively, the implant 12 can be used like in the above example of the rotator cuff, if the tear in the tendon is in a proper location for such a repair.

Other further injuries may benefit from the use of implant 12, such as in the repair of muscle or repairs to soft tissue in, for example, the spine.

Instrumentation or imaging techniques to measure and match the tissue defect and/or surgical instruments used in conjunction with implant implantation may be packaged with the implant as a kit.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

The entire disclosure of all references discussed herein is hereby incorporated by reference herein.

1. An implant for repairing a tissue defect, comprising a porous material, wherein said material is expandable or compressible.

2. The implant of claim 1, wherein said porous material allows tissue ingrowth.

3. The implant of claim 1, wherein said material comprises a scaffold.

4. The implant of claim 1, wherein said material comprises collagen or demineralized bone matrix.

5. The implant of claim 1, wherein said material comprises a material selected from the group consisting of autologous tissue, allogenic tissue, xenogenic tissue, and synthetic material.

6. The implant of claim 1, wherein said material is expandable by at least 5% by volume.

7. The implant of claim 1, wherein said material is compressible by at least 5% by volume.

8. A method for the repair of a tissue defect in a patient in need thereof, comprising implanting at least one implant between two adjacent tissues, wherein said implant comprises a porous material, wherein said implant is expandable or compressible.

9. The method of claim 8, wherein said two tissues comprise bone and a soft tissue selected from the group consisting of tendons, ligaments, fibrous tissues, meniscus tissue, muscle, cartilage, skin, dermal layers, joint capsule, and synovium.

10. The method of claim 9, further comprising reattaching said soft tissue to said bone, wherein said adjacent two tissues comprise a defect, wherein said defect is a connection site between bone and soft tissue selected from the group consisting of tendons, ligaments, fibrous tissues, meniscus tissue, muscle, cartilage, skin, dermal layers, joint capsule, and synovium.

11. The method of claim 8, wherein said two tissues comprise soft tissue selected from the group consisting of tendons, ligaments, fibrous tissues, meniscus tissue, muscle, cartilage, skin, dermal layers, joint capsule, and synovium.

12. The method of claim 8, wherein said implant is placed within said defect such that the adjacent tissues integrate through the implant.

13. The method of claim 8, wherein said material comprises a scaffold.

14. The method of claim 8, wherein said material comprises a material selected from the group consisting of collagen and demineralized bone matrix.

15. The method of claim 8, wherein said material comprises a material selected from the group consisting of autologous tissue, allogenic tissue, xenogenic tissue, and synthetic material.

16. The method of claim 8, wherein said implant is placed in between the two tissues such that at least one of the two tissues integrate through said implant.

17. The implant of claim 8, wherein the step of implanting an implant further comprises implanting multiple implants between two adjacent tissues.

18. The implant of claim 9, wherein said implant further comprises cells selected from the group consisting of embryonic stem cells, stem cells, bone marrow cells, mesenchymal cells, synovium cells, synovial fluid cells, progenitor cells, chondroblasts, chondrocytes, and osteoblasts.
